(19) United States
(12) Patent Application Publication
(10) Pub. No.: US 2006/0239935 A1
(43) Pub. Date: Oct. 26, 2006

(54) COMPOSITIONS FOR INHALATION

(75) Inventors: Thierry Bouyssou, Birkenhard (DE); Ingo Konetzki, Warthausen (DE); Michael P. Pieper, Biberach (DE); Andreas Schnapp, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/379,713

(22) Filed: Apr. 21, 2006

(30) Foreign Application Priority Data
Apr. 23, 2005 (EP) 05008957

(51) Int. Cl. A61K 31/4745; A61K 31/4704; A61K 9/14; A61L 9/04

(52) U.S. Cl. 424/46; 514/291; 514/312; 514/305

(57) ABSTRACT

The present invention relates to new pharmaceutical compositions for inhalation containing one or more, preferably one anticholinergic 1 in combination with one or more pharmacologically acceptable acid addition salts of a compound of formula 2′, 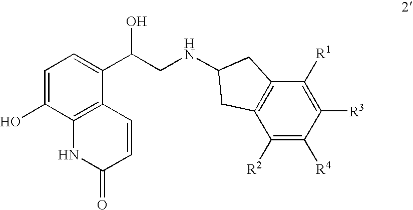
wherein the groups R¹, R², R³ and R⁴ may have the meanings given in the specification and in the claims, processes for preparing them and their use in the treatment of respiratory complaints.

### RELATED APPLICATION

This application claims priority to European Patent Application No. 05 008 957, filed Apr. 23, 2005, the content of which is incorporated herein by reference in its entirety.

### FIELD OF THE INVENTION

The present invention relates to new pharmaceutical compositions for inhalation containing one or more, preferably one anticholinergic 1 in combination with one or more pharmacologically acceptable acid addition salts of a compound of formula 2′, 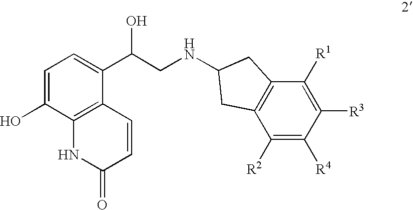
wherein the groups R¹, R², R³ and R⁴ may have the meanings given in the specification and claims, processes for preparing them and their use in the treatment of respiratory complaints.

### BRIEF DESCRIPTION OF THE DRAWINGS

FIG. 1 illustrates an exploded view of a preferred inhaler for administration of the pharmaceutical compositions described herein.

### DETAILED DESCRIPTION OF THE INVENTION

Surprisingly, an unexpectedly beneficial therapeutic effect can be observed in the treatment of inflammatory or obstructive diseases of the respiratory tract if one or more, preferably one, anticholinergic 1 is used in conjunction with pharmacologically acceptable salts of a betamimetic of formula 2′ 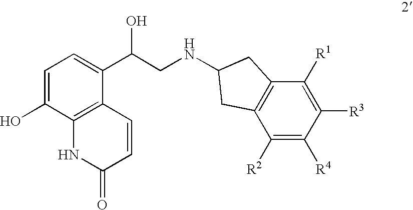
wherein the groups R¹, R², R³ and R⁴ may have the meanings given hereinafter.

This may significantly reduce undesirable side effects, such as are frequently observed when β-mimetics are administered to humans. The central side effects of β-mimetics include for example general malaise, excitement, sleeplessness, anxiety, trembling fingers, sweats and headaches.

Accordingly, the present invention relates to pharmaceutical combinations characterised in that they contain one or more, preferably one anticholinergic 1 combined with a pharmacologically acceptable salt of a compound of formula 2′ 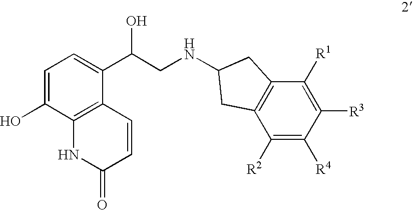
wherein
R¹ and R² which may be identical or different denote hydrogen or C₁-C₄-alkyl;
R³ and R⁴ which may be identical or different denote hydrogen, C₁-C₄-alkyl, —O-C₁-C₄-alkyl, —C₁-C₄-alkylene-O—C₁-C₄-alkyl or
   R³ and R⁴ together denote one of the bridging groups —C₁-C₄-alkylene or —O—C₁-C₄-alkylene-O.

The anticholinergic 1 which may be used according to the invention is preferably a salt of formula 1a, 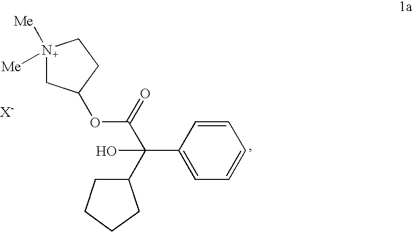
wherein
X⁻ denotes an anion with a single negative charge, preferably an anion selected from among fluoride, chloride, bromide, iodide, sulphate, phosphate, methanesulphonate, nitrate, maleate, acetate, citrate, fumarate, tartrate, oxalate, succinate, benzoate and p-toluenesulphonate, optionally in the form of the racemates, enantiomers or hydrates thereof,
   optionally in the form of the diastereomers, mixtures of the diastereomers or racemates thereof, and optionally in the form of the hydrates and/or solvates thereof.

Preferred pharmaceutical combinations contain salts of formula 1a, wherein
X⁻ denotes an anion with a single negative charge, preferably an anion selected from among fluoride, chloride, bromide, methanesulphonate and p-toluenesulphonate, preferably bromide,
   optionally in the form of the diastereomers, mixtures of the diastereomers or racemates thereof, and optionally in the form of the hydrates and/or solvates thereof.

Preferred pharmaceutical combinations contain salts of formula 1a, wherein
X⁻ denotes an anion with a single negative charge, preferably an anion selected from among chloride, bromide and methanesulphonate, preferably bromide,
   optionally in the form of the diastereomers, mixtures of the diastereomers or racemates thereof, and optionally in the form of the hydrates and/or solvates thereof.

The compound of formula 1a may particularly preferably be contained in the pharmaceutical combinations according to the invention in the form of one of the 4 diastereomers thereof, which are listed below: 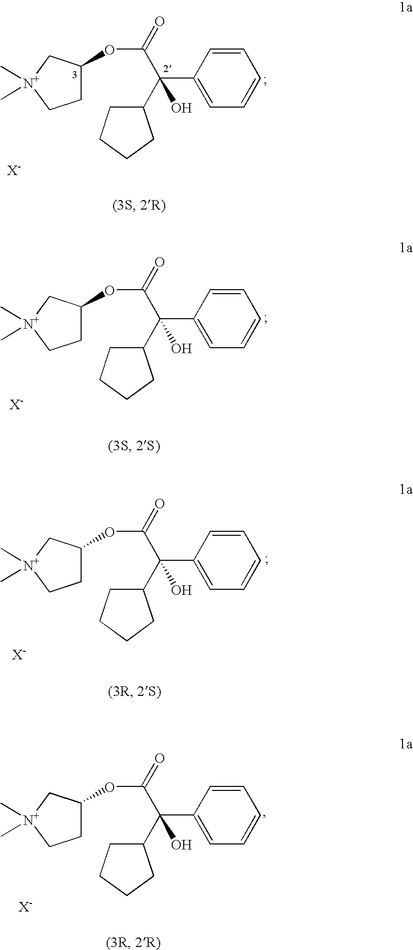
while the anion X— may have the meanings given above.

Of the above-mentioned diastereomers the (3R,2′R)-diastereomer according to the invention is of particular importance. Methods for preparing the above-mentioned diastereomerically pure compounds are disclosed for example in WO98/21183.

Particularly preferred drug combinations contain the compound of formula 1a in the form of the bromides, optionally in the form of the diastereomers, mixtures of diastereomers or racemates thereof, and optionally in the form of the hydrates and/or solvates thereof.

The anticholinergic 1 according to the invention may also preferably consist of a salt of formula 1b, 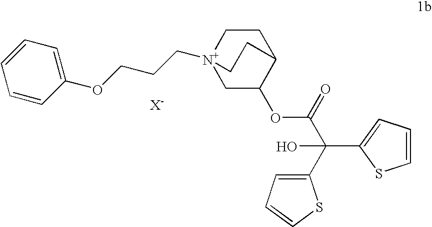
wherein
X⁻ denotes an anion with a single negative charge, preferably an anion selected from among fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the enantiomers, mixtures of enantiomers or racemates thereof, and optionally in the form of the hydrates and/or solvates thereof.

Preferred pharmaceutical combinations contain salts of formula 1b, wherein
X⁻ denotes an anion with a single negative charge, preferably an anion selected from among fluoride, chloride, bromide, methanesulphonate and p-toluenesulphonate, preferably bromide,
   optionally in the form of the enantiomers, mixtures of enantiomers or racemates thereof, and optionally in the form of the hydrates and/or solvates thereof.

Preferred pharmaceutical combinations contain salts of formula 1b, wherein
X⁻ denotes an anion with a single negative charge, preferably an anion selected from among chloride, bromide and methanesulphonate, preferably bromide,
   optionally in the form of the enantiomers, mixtures of enantiomers or racemates thereof, and optionally in the form of the hydrates and/or solvates thereof.

Particularly preferred pharmaceutical combinations contain the compound of formula 1b in the form of the bromides, optionally in the form of the enantiomers, mixtures of enantiomers or racemates thereof, and optionally in the form of the hydrates and/or solvates thereof.

Of particular importance are those drug combinations which contain enantiomers of formula 1b-en 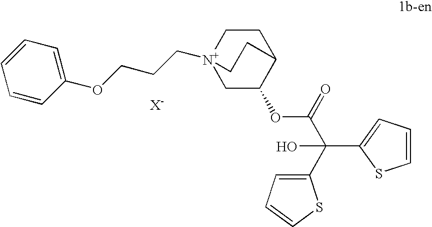
wherein X⁻ may have the above-mentioned meanings.

In another preferred embodiment of the present invention the anticholinergics 1 contained in the drug combinations according to the invention are selected from the compounds of formula 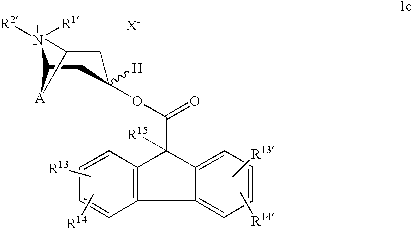
wherein
X⁻ may have the above-mentioned meanings and wherein
A denotes a double-bonded group selected from the groups 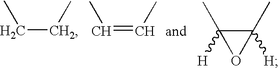
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen,
   or
   R^{1′} and R^{2′} together denote a —C₃-C₅-alkylene bridge;
      R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen.

The compounds of formula 1c are known in the prior art (WO 03/064419).

Within the scope of the drug combinations according to the invention particularly preferred compounds of formula 1c are those wherein
A denotes a double-bonded group selected from 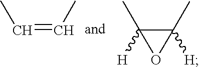
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the drug combinations according to the invention particularly preferred compounds of formula 1c are those wherein
A denotes a double-bonded group selected from 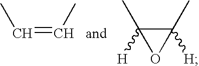
X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those drug combinations which contain one of the following compounds of formula 1c:
tropenol 9-hydroxy-fluorene-9-carboxylate methobromide (1c.1);
tropenol 9-fluoro-fluorene-9-carboxylate methobromide (1c.2);
scopine 9-hydroxy-fluorene-9-carboxylate methobromide (1c.3);
scopine 9-fluoro-fluorene-9-carboxylate methobromide (1c.4);
tropenol 9-methyl-fluorene-9-carboxylate methobromide (1c.5);
scopine 9-methyl-fluorene-9-carboxylate methobromide (1c.6);

The compounds of formula 1c may optionally be present in the form of the enantiomers, mixtures or enantiomers or racemates thereof, and optionally in the form of the hydrates and/or solvates thereof.

In another preferred embodiment of the present invention the anticholinergics 1 contained in the drug combinations according to the invention are preferably selected from the compounds of formula 1d 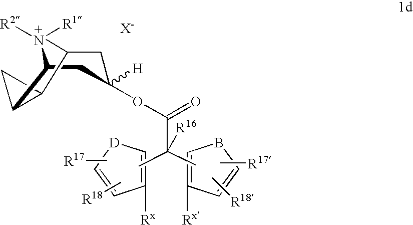
wherein X⁻ may have the meanings given above and wherein
D and B which may be identical or different, preferably identical, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2′} which may be identical or different, denote —C₁-C₅-alkyl, which may optionally be substituted by —C₃-C₆-cycloalkyl, hydroxy or halogen,
   or
   R^{1″} and R^{2″} together denote a —C₃-C₅-alkylene bridge;
      R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen,
   or
   R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 1d are known from the prior art (WO 03/064418).

Within the scope of the drug combinations according to the invention preferred compounds of formula 1d are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁-C₄-alkyl, —C₁-C₄-alkyloxy, —CF₃, —CHF₂,
   fluorine, chlorine or bromine;
R^{1″} and R^{2″} which may be identical or different, denote C₁-C₄-alkyl, which may optionally be substituted by hydroxy, fluorine, chlorine or bromine, or
   R^{1″} and R^{2″} together denote a —C₃-C₄-alkylene bridge;
R¹⁷, R¹⁸, R^{17′} and R^{18′} which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine,
   or
   R^{x} and R^{x′} together denote a single bond or a double-bonded group selected from O, S, NH— and CH₂.

Within the scope of the drug combinations according to the invention particularly preferred compounds of formula 1d are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen, or
   R^{x} and R^{x′} together denote a single bond or —O.

Within the scope of the drug combinations according to the invention other particularly preferred compounds of formula 1d are those wherein
X⁻ denotes bromide;
D and B denotes —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen, or
   R^{x} and R^{x′} together denote a single bond or the group —O.

Of particular importance are those drug combinations which contain one of the following compounds of formula 1d:
cyclopropyltropine benzilate methobromide (1d.1);
cyclopropyltropine 2,2-diphenylpropionate methobromide (1d.2);
cyclopropyltropine 9-hydroxy-xanthene-9-carboxylate methobromide (1d.3);
cyclopropyltropine 9-methyl-fluorene-9-carboxylate methobromide (1d.4);
cyclopropyltropine 9-methyl-xanthene-9-carboxylate methobromide (1d.5);
cyclopropyltropine 9-hydroxy-fluorene-9-carboxylate methobromide (1d.6);
methyl cyclopropyltropine 4,4′-difluorobenzilate methobromide (1d.7).

The compounds of formula 1d may optionally be present in the form of the enantiomers, mixture of enantiomers or racemates thereof and optionally in the form of the hydrates and/or solvates thereof.

In another preferred embodiment of the present invention the anticholinergics 1 contained in the drug combinations according to the invention are selected from the compounds of formula 1e 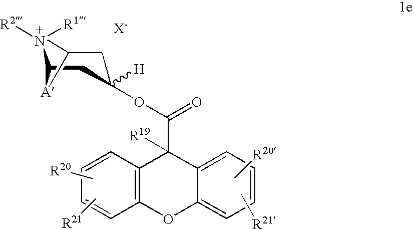
wherein X⁻ may have the meanings given above and wherein
A′ denotes a double-bonded group selected from 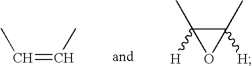
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen,
   or
   R^{1′″} and R^{2′″} together denote a —C₃-C₅-alkylene bridge;
      R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 1e are known from the prior art (WO 03/064417).

Within the scope of the drug combinations according to the invention preferred compounds of formula 1e are those wherein
A′ denotes a double-bonded group selected from 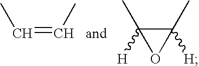
X⁻ denotes chloride, bromide or methanesulphonate, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the drug combinations according to the invention particularly preferred compounds of formula 1e are those wherein
A′ denotes a double-bonded group selected from 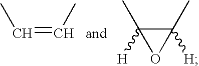
X⁻ denotes bromide;
R¹⁹ denotes hydroxy or methyl, preferably methyl;
R^{1′″} and R^{2′″} which may be identical or different, denote methyl or ethyl, preferably methyl;
R³, R⁴, R^{3′} and R^{4′} which may be identical or different, denote hydrogen or fluorine.

Of particular importance are those drug combinations which contain one of the following compounds of formula 1e:
tropenol 9-hydroxy-xanthene-9-carboxylate methobromide (1e.1);
scopine 9-hydroxy-xanthene-9-carboxylate methobromide (1e.2);
tropenol 9-methyl-xanthene-9-carboxylate methobromide (1e.3);
scopine 9-methyl-xanthene-9-carboxylate methobromide (1e.4);
tropenol 9-ethyl-xanthene-9-carboxylate methobromide (1e.5);
tropenol 9-difluoromethyl-xanthene-9-carboxylate methobromide (1e.6);
scopine 9-hydroxymethyl-xanthene-9-carboxylate methobromide (1e.7).

The compounds of formula 1e may optionally be present in the form of the enantiomers, mixture of enantiomers or racemates thereof and optionally in the form of the hydrates and/or solvates thereof.

Within the scope of the present invention any reference to anticholinergics 1′ is to be taken as a reference to the pharmacologically active cations of the various salts. These cations may be represented by the following formulae:

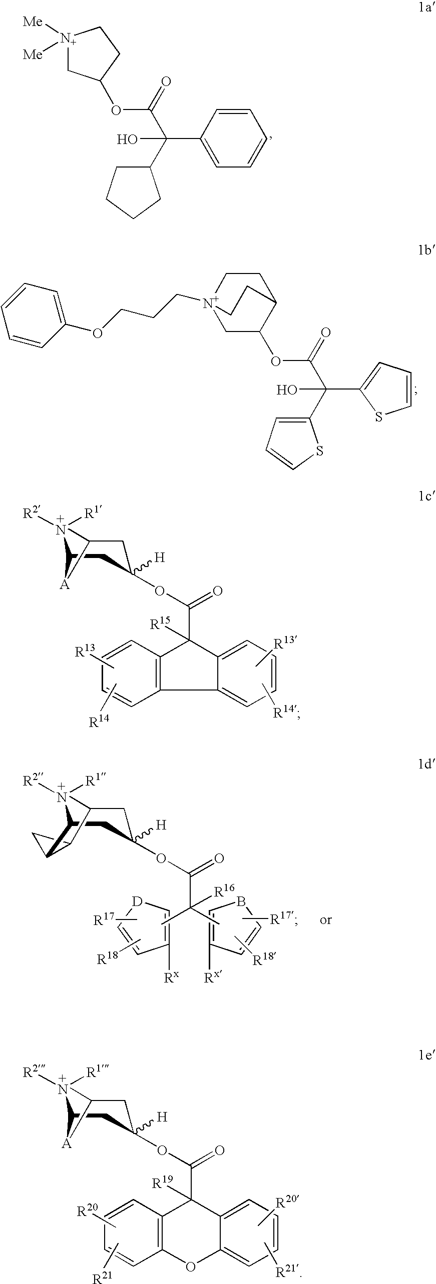

In another preferred embodiment of the present invention the anticholinergics 1 contained in the drug combinations according to the invention are selected from among oxitropium salts (1f), flutropium salts (1g), ipratropium salts (1h) and trospium salts (1i). In the above-mentioned salts 1f to 1i the cations oxitropium, flutropium, ipratropium and trospium are pharmacologically active ingredients. Explicit references to the above-mentioned cations are indicated by the numerals 1f′ to 1i′. Each reference to the above-mentioned salts 1f to 1i naturally includes a reference to the corresponding cations. By the salts 1f to 1i are meant according to the invention those compounds which contain in addition to the cations oxitropium (1f′), flutropium (1g′), ipratropium (1h′) and trospium (1i′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate contain, while the chloride, bromide, iodide, sulphate, methanesulphonate or p-toluenesulphonate are preferred as counter-ions. Of all the salts the chloride, bromide, iodide and methanesulphonate are particularly preferred. In the case of the trospium salts (1i) the chloride is particularly preferred. Of the other salts 1f to 1h the methanesulphonates and bromides are of particular importance. Of particular importance are medicament combinations which contain oxitropium salts (1f) or ipratropium salts (1h), while the respective bromides are particularly important according to the invention. The above-mentioned salts may optionally be present in the medicament combinations according to the invention in the form of their solvates or hydrates, preferably in the form of their hydrates.

Preferably in the combinations according to the invention salts of the compounds of formula 2′ are used wherein
R¹ and R² which may be identical or different denote hydrogen, methyl or ethyl;
R³ and R⁴ which may be identical or different denote hydrogen, methyl, ethyl, propyl, butyl, methoxy, ethoxy, methyoxymethyl, or methoxyethyl, or
   R³ and R⁴ together denote one of the bridging groups propylene, butylene, —O-ethylene-O or —O-propylene-O—.

Particularly preferably in the combinations according to the invention salts of the compounds of formula 2′ are used wherein
R¹ and R² which may be identical or different denote hydrogen or ethyl, preferably hydrogen;
R³ and R⁴ which may be identical or different denote hydrogen, methyl, ethyl, propyl, butyl or methyoxymethyl or
   R³ and R⁴ together denote one of the bridging groups butylene or —O-ethylene-O—.

Particularly preferably according to the invention compounds of formula 2′ are used in the combinations according to the invention, wherein
a) R¹ and R² denote hydrogen and R³ and R⁴ denote ethyl (2a′); or
b) R¹ and R² denote hydrogen and R³ and R⁴ denote methyl (2b′); or
c) R¹ and R² denote ethyl and R³ and R⁴ denote hydrogen (2c′); or
d) R¹ and R² denote hydrogen and R³ and R⁴ together denote butylene (2d′); or
e) R¹ and R² denote hydrogen and R³ and R⁴ together denote —O-ethylene-O-(2e′); or
f) R¹ and R² denote hydrogen and R³ and R⁴ denote tert-butyl (2f′); or
g) R¹ and R² denote hydrogen and R³ and R⁴ denote iso-propyl (2g′); or
h) R¹ and R² denote hydrogen and R³ and R⁴ denote methoxymethyl (2h′).

Of the above-mentioned compounds the structure defined under a), wherein R¹ and R² denote hydrogen and R³ and R⁴ denote ethyl, is of exceptional importance in the drug combinations according to the invention. The acid addition salts of this compound are hereinafter also referred to as compounds 2a, while a reference to the free base of this compound is characterised by the name 2a′ according to the following formula

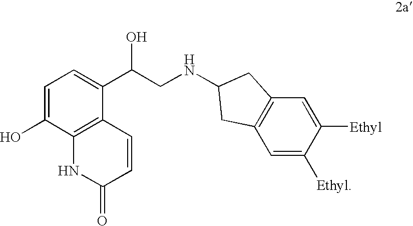

The salts 2 of the compounds of formula 2′ may be used in the pharmaceutical combinations according to the invention in the form of the racemates, enantiomers or mixtures thereof. The separation of enantiomers from the racemates may be carried out using methods known in the art (e.g. by chromatography on chiral phases, etc.). If the salts of the compounds of formula 2′ are used in the form of their enantiomers it is particularly preferable to use the enantiomers which are R-configured at the C—OH group.

Unless otherwise stated, the term alkyl groups denotes branched and unbranched alkyl groups with 1 to 4 carbon atoms. The following are mentioned by way of example: methyl, ethyl, propyl or butyl. The abbreviations Me, Et, Prop or Bu may optionally also be used to denote the groups methyl, ethyl, propyl or butyl. Unless stated otherwise, the definitions propyl and butyl include all the possible isomeric forms of the groups in question. Thus, for example, propyl includes n-propyl and iso-propyl, butyl includes iso-butyl, sec. butyl and tert-butyl etc.

Unless otherwise stated, the term alkylene groups denotes branched and unbranched double-bonded alkyl bridges with 1 to 4 carbon atoms. The following are mentioned by way of example: methylene, ethylene, propylene or butylene.

Unless otherwise stated, the term alkyloxy groups (also known as —O—C₁-C₄-alkyl groups) denotes branched and unbranched alkyl groups with 1 to 4 carbon atoms which are linked by an oxygen atom. The following are mentioned by way of example: methyloxy, ethyloxy, propyloxy or butyloxy. The abbreviations MeO, EtO, PropO or BuO— may optionally also be used to denote the groups methyloxy, ethyloxy, propyloxy or butyloxy. Unless stated otherwise, the definitions propyloxy and butyloxy include all the possible isomeric forms of the groups in question. Thus, for example, propyloxy includes n-propyloxy and iso-propyloxy, butyloxy includes iso-butyloxy, sec. butyloxy and tert.-butyloxy etc. In some cases, the term alkoxy is used within the scope of the present invention instead of the term alkyloxy. Accordingly, the terms methoxy, ethoxy, propoxy or butoxy may optionally be used to denote the groups methyloxy, ethyloxy, propyloxy or butyloxy.

Unless otherwise stated, the term alkylene-alkyloxy groups denotes branched and unbranched double-bonded alkyl bridges with 1 to 4 carbon atoms which are mono-, di- or trisubstituted, preferably monosubstituted, by an alkyloxy group.

Within the scope of the present invention any reference to compounds 2 is to be taken as being a reference to physiologically acceptable acid addition salts. Examples of physiologically acceptable acid addition salts 2 according to the invention are pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid or maleic acid. The salts 2 may also be prepared using mixtures of the above-mentioned acids.

Preferably, according to the invention, the salts 2 are selected from among the hydrochloride, hydrobromide, sulphate, phosphate, fumarate, methanesulphonate and maleate. Particularly preferably, in the case of the compound 2a′, the salts 2 are selected from hydrochloride and maleate, of which the maleate is particularly preferred.

If within the scope of the present invention there is a reference to compounds of formula 2′, which are not in the salt form this is indicated by the numeral 2′ whereas a reference to 2 is to be taken as a reference to the acid addition salts of a compound of formula 2′. The compound of formula 2′ and 2 and processes for the preparation thereof are known from WO 00/75114, to which reference is hereby made.

The combinations of active substances according to the invention have advantageous properties and are characterised partly by a rapid onset of activity and also by a long-lasting effect. This is of great importance to the wellbeing of the patient as on the one hand he experiences a rapid improvement in his condition after the combination has been administered and also thanks to the long-lasting effect it is sufficient to take the drug once a day.

The effects mentioned above may be observed both when the two active substances are administered simultaneously in a single active substance formulation and when they are administered successively in separate formulations. According to the invention, it is preferable to administer the two active substance ingredients simultaneously in a single formulation.

In one aspect the present invention relates to a pharmaceutical composition which contains one or more anticholinergics 1 and salts 2 of a compound of formula 2′ optionally in the form of the solvates or hydrates thereof. The active substances may be present together in a single formulation or in two separate formulations. Pharmaceutical compositions which contain the active substances 1 and 2 in a single formulation are preferred according to the invention.

In one aspect the present invention relates to the above-mentioned drug combinations which contain a pharmaceutically acceptable carrier in addition to therapeutically effective amounts of 1 and 2. In one aspect the present invention relates to the above-mentioned pharmaceutical compositions which do not contain any pharmaceutically acceptable carrier in addition to therapeutically effective amounts of 1 and 2.

The present invention further relates to the use of therapeutically effective amounts of the active substances 1 for preparing a medicament also containing one or more, preferably one active substance 2, for the treatment of inflammatory and obstructive respiratory complaints, for inhibiting premature labour in midwifery (tocolysis), for restoring sinus rhythm in the heart in cases of atrio-ventricular block, for correcting bradycardiac heart rhythm disorders (antiarrhythmic agent), for the treatment of circulatory shock (vasodilatation and increasing the heart-time volume) as well as the treatment of itching and skin inflammation.

In a preferred aspect the present invention relates to the use of therapeutically effective amounts of the active substances 1 for preparing a medicament also containing one or more, preferably one active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic scleroderma or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention further relates to the use of therapeutically effective amounts of an active substance of formula 1 in combination with effective amounts of an active substance 2 for preparing a pharmaceutical composition for the treatment of one of the above-mentioned diseases.

The present invention further relates to a method of treating one of the above-mentioned diseases, which is characterised in that therapeutically effective amounts of an active substance of formula 1 are administered in combination with therapeutically effective amounts of an active substance 2.

The ratios in which the two active substances 1 and 2 may be used in the active substance combinations according to the invention are variable. The active substances 1 and 2 may optionally be used in the form of the solvates or hydrates thereof. Depending on the choice of the salts 1 and 2 the weight ratios which may be used within the scope of the present invention vary due to the different molecular weights of the different salt forms. Therefore the weight ratios specified below are based on the cations 1′ and the free bases 2′.

Examples of preferred pharmaceutical combinations according to the invention are combinations containing the compounds 1a and 2a; 1a and 2b; 1a and 2c; 1a and 2d; 1a and 2e; 1a and 2f; 1a and 2g; 1a and 2h. In the above-mentioned drug combinations it is particularly preferable according to the invention to use the (3R,2′R)-enantiomer of compound 1a.

Examples of preferred drug combinations according to the invention are combinations containing the compounds 1b and 2a; 1b and 2b; 1b and 2c; 1b and 2d; 1b and 2e; 1b and 2f; 1b and 2g; 1b and 2h. In the above-mentioned drug combinations it is particularly preferable according to the invention to use the enantiomer of formula 1b-en.

Examples of other preferred drug combinations according to the invention are combinations containing the compounds 1c.1 and 2a; 1c.1 and 2b; 1c.1 and 2c; 1c.1 and 2d; 1c.1 and 2e; 1c.1 and 2f; 1c.1 and 2g; 1c.1 and 2h; 1c.2 and 2a; 1c.2 and 2b; 1c.2 and 2c; 1c.2 and 2d; 1c.2 and 2e; 1c.2 and 2f; 1c.2 and 2g; 1c.2 and 2h; 1c.3 and 2a; 1c.3 and 2b, 1c.3 and 2c; 1c.3 and 2d; 1c.3 and 2e; 1c.3 and 2f; 1c.3 and 2g; 1c.3 and 2h; 1c.4 and 2a; 1c.4 and 2b: 1c.4 and 2c; 1c.4 and 2d; 1c.4 and 2e; 1c.4 and 2f; 1c.4 and 2g; 1c.4 and 2h; 1c.5 and 2a; 1c.5 and 2b: 1c.5 and 2c; 1c.5 and 2d; 1c.5 and 2e; 1c.5 and f; 1c.5 and 2g; 1c.5 and 2h; 1c.6 and 2a; 1c.6 and 2b: 1c.6 and 2c; 1c.6 and 2d; 1c.6 and 2e; 1c.6 and 2f; 1c.6 and 2g; 1c.6 and 2h.

Examples of other preferred drug combinations according to the invention are combinations containing the compounds 1d.1 and 2a; 1d.1 and 2b; 1d.1 and 2c; 1d.1 and 2d; 1d.1 and 2e; 1d.1 and 2f; 1d.1 and 2g; 1d.1 and 2h; 1d.2 and 2a; 1d.2 and 2b; 1d.2 and 2c; 1d.2 and 2d; 1d.2 and 2e; 1d.2 and 2f; 1d.2 and 2g; 1d.2 and 2h; 1d.3 and 2a; 1d.3 and 2b; 1d.3 and 2c; 1d.3 and 2d; 1d.3 and 2e; 1d.3 and 2f; 1d.3 and 2g; 1d.3 and 2h; 1d.4 and 2a; 1d.4 and 2b; 1d.4 and 2c; 1d.4 and 2d; 1d.4 and 2e; 1d.4 and 2f; 1d.4 and 2g; 1d.4 and 2h; 1d.5 and 2a; 1d.5 and 2b; 1d.5 and 2c; 1d.5 and 2d; 1d.5 and 2e; 1d.5 and 2f; 1d.5 and 2g; 1d.5 and 2h; 1d.6 and 2a; 1d.6 and 2b; 1d.6 and 2c; 1d.6 and 2d; 1d.6 and 2e; 1d.6 and 2f; 1d.6 and 2g; 1d.6 and 2h; 1d.7 and 2a; 1d.7 and 2b; 1d.7 and 2c; 1d.7 and 2d; 1d.7 and 2e; 1d.7 and 2f; 1d.7 and 2g; 1d.7 and 2h.

Examples of other preferred drug combinations according to the invention are combinations containing the compounds 1e.1 and 2a; 1e.1 and 2b 1e.1 and 2c; 1e.1 and 2d; 1e.1 and 2e; 1e.1 and 2f; 1e.1 and 2g; 1e.1 and 2h; 1e.2 and 2a; 1e.2 and 2b; 1e.2 and 2c; 1e.2 and 2d; 1e.2 and 2e; 1e.2 and 2f; 1e.2 and 2g; 1e.2 and 2h; 1e.3 and 2a; 1e.3 and 2b; 1e.3 and 2c; 1e.3 and 2d; 1e.3 and 2e; 1e.3 and 2f; 1e.3 and 2g; 1e.3 and 2h; 1e.4 and 2a; 1e.4 and 2b; 1e.4 and 2c; 1e.4 and 2d; 1e.4 and 2e; 1e.4 and 2f; 1e.4 and 2g; 1e.4 and 2h; 1e.5 and 2a; 1e.5 and 2b; 1e.5 and 2c; 1e.5 and 2d; 1e.5 and 2e; 1e.5 and 2f; 1e.5 and 2g; 1e.5 and 2h; 1e.6 and 2a; 1e.6 and 2b; 1e.6 and 2c; 1e.6 and 2d; 1e.6 and 2e; 1e.6 and 2f; 1e.6 and 2g; 1e.6 and 2h; 1e.7 and 2a; 1e.7 and 2b; 1e.7 and 2c; 1e.7 and 2d; 1e.7 and 2e; 1e.7 and 2f; 1e.7 and 2g; 1e.7 and 2h.

Examples of other preferred drug combinations according to the invention are combinations containing the compounds 1f and 2a; 1f and 2b; 1f and 2c; 1f and 2d; 1f and 2e; 1f and 2f; 1f and 2g; 1f and 2h.

Examples of other preferred drug combinations according to the invention are combinations containing the compounds 1g and 2a; 1g and 2b; 1g and 2c; 1g and 2d; 1g and 2e; 1g and 2f; 1g and 2g; 1g and 2h.

Examples of other preferred drug combinations according to the invention are combinations containing the compounds 1h and 2a; 1h and 2b; 1h and 2c; 1h and 2d; 1h and 2e; 1h and 2f; 1h and 1g; 1h and 2h.

Examples of other preferred drug combinations according to the invention are combinations containing the compounds 1i and 2a; 1i and 2b; 1i and 2c; 1i and 2d; 1i and 2e; 1i and 2f; 1i and 2g; 1i and 2h.

The active substance combinations according to the invention may contain 1′ and 2′ in ratios by weight which are in the range from about 1:30 to 400:1, preferably 1:25 to 200:1, preferably 1:20 to 100:1, most preferably from 1:15 to 50:1, for example.

For example and without restricting the scope of the invention thereto, preferred combinations of 1 and 2 according to the invention may contain the cation 1′ and the compound 2′ in the following ratios by weight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1.

The pharmaceutical compositions according to the invention containing the combinations of 1 and 2 are usually administered such that each single dose contains the cation 1′ and the compound 2 together in dosages of 0.01 to 10000 μg, preferably 0.1 to 5000 μg, preferably 5 to 2000 μg, particularly preferably 15 to 1000 μg, more preferably 20 to 800 μg, and preferably according to the invention 30 to 750 μg, preferably 40 to 700 μg.

For example combinations of 1 and 2 according to the invention contain an amount of 1′ and 2′ such that the total dosage per single dose is about 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 g, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg, 1105 μg, 1110 μg, 1115 μg, 1120 μg, 1125 μg, 1130 μg, 1135 μg, 1140 μg, 1145 μg, 1150 μg, 1155 μg, 1160 μg, 1165 μg, 1170 μg, 1175 μg, 1180 μg, 1185 μg, 1190 μg, 1195 μg, 1200 μg, 1205 μg, 1210 μg, 1215 μg, 1220 μg, 1225 μg, 1230 μg, 1235 μg, 1240 μg, 1245 μg, 1250 μg, 1255 μg, 1260 μg, 1265 μg, 1270 μg, 1275 μg, 1280 μg, 1285 μg, 1290 μg, 1295 μg, 1300 μg, 1305 μg, 1310 μg, 1315 μg, 1320 μg, 1325 μg, 1330 μg, 1335 μg, 1340 μg, 1345 μg, 1350 μg, 1355 μg, 1360 μg, 1365 μg, 1370 μg, 1375 μg, 1380 μg, 1385 μg, 1390 μg, 1395 μg, or similar. It is clear to the skilled man that the proposed dosages per single dose as stated above are not to be regarded as being restricted to the explicit values given. Fluctuations of about ±2.5 μg, particularly fluctuations in the decimal range are also included, as is apparent to anyone skilled in the art. In these dosage ranges, the active substances 1′ and 2′ are present in the weight ratios described hereinbefore.

Within the scope of the drug combinations according to the invention 0.1-1000 μg of a compound of formula 1 may be administered per single dose, for example. Preferably 1-500 μg, particularly preferably 3-100 μg of the compound of formula 1 are administered per single dose, while a dosage range of 5-75 μg, preferably 7-50 μg is preferred, according to the invention. Particularly preferably, the pharmaceutical compositions according to the invention are administered in an amount such that 9-40 μg, particularly preferably 11-30 μg, more preferably 12-25 μg of the compound of formula 1 are administered per single dose. For example, and without restricting the scope of the invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

Without restricting the scope of the invention thereto, in the case of the cation 1a′ amounts of anticholinergic may be administered such that each single dose contains 1-500 μg, preferably 5-300 μg, particularly preferably 15-200 μg. For example and without restricting the present invention thereto, each single dose may contain 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1a′. The corresponding amount of salt 1a used or optionally hydrate or solvate used are easily calculated by the skilled man depending on the choice of the anion. In the case of glycopyrronium 1a′ the dosages specified above are preferably administered one to four times a day, while administration twice to three times a day is particularly preferred according to the invention. If the preferred diastereomer (3R,2′R)-1a is used according to the invention, the above-mentioned quantities of active substance may optionally also be administered once a day.

Without restricting the scope of the invention thereto, in the case of the cation 1b amounts of anticholinergic are administered such that each single dose contains 50-1000 μg, preferably 100-800 μg, particularly preferably 200-700 μg, particularly preferably 300-600 μg of 1b′. For example and without restricting the present invention thereto, each single dose may contain 300 μg, 350 μg, 400 μg, 450 μg, 500 μg, 550 μg, or 600 μg 1b′. The corresponding amount of salt 1b or optionally hydrates or solvates used are easily calculated by the skilled man depending on the choice of the anion. In the case of the cation 1b′ the dosages specified above are preferably administered once to three times a day, while administration once or twice, most preferably once a day particularly when administering the enantiomerically pure compounds 1b-en is particularly preferred according to the invention.

Without restricting the scope of the invention thereto, in the case of the cations 1c′ to 1e′ amounts of anticholinergic (1c′, 1d′ or 1e′) are administered such that each single dose contains 1-500 μg, preferably 5-300 μg, particularly preferably 10-200 μg of 1c′, 1d′ or 1e′. For example and without restricting the present invention thereto, each single dose may contain 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1c′, 1d′ or 1e′. The corresponding amount of salt 1c, 1d or 1e or optionally hydrates or solvates used are easily calculated by the skilled man depending on the choice of the anion.

In the case of the cations 1c, 1d or 1e the dosages specified above are preferably administered once to three times a day, while administration once or twice, most preferably once a day is particularly preferred according to the invention.

Without restricting the scope of the invention thereto, in the case of the cation 1f′ amounts of anticholinergic (1f′) may be administered such that each single dose contains 1-500 μg, preferably 5-300 μg, particularly preferably 15-200 μg of 1f′. For example and without restricting the present invention thereto, each single dose may contain 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1f′. The corresponding amount of salt 1f or optionally hydrates or solvates used are easily calculated by the skilled man depending on the choice of the anion. In the case of oxitropium 1f′ the dosages specified above are preferably administered one to four times a day, while administration twice to three times a day is particularly preferred according to the invention.

Without restricting the scope of the invention thereto, may in the case of the cation 1g′ amounts of anticholinergic (1g′) may be administered such that each single dose contains 1-500 μg, preferably 5-300 μg, particularly preferably 15-200 μg of 1g′. For example and without restricting the present invention thereto, each single dose may contain 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1g′. The corresponding amount of salt 1g or optionally hydrates or solvates used are easily calculated by the skilled man depending on the choice of the anion. In the case of flutropium 1g′ the dosages specified above are preferably administered one to four times a day, while administration twice to three times a day is particularly preferred according to the invention.

Without restricting the scope of the invention thereto, in the case of the cation 1h′ amounts of anticholinergic (1h′) may be administered such that each single dose contains 1-500 μg, preferably 5-300 μg, particularly preferably 20-200 μg of 1h′. For example and without restricting the present invention thereto, each single dose may contain 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1h′. The corresponding amount of salt 1h or optionally hydrates or solvates used are easily calculated by the skilled man depending on the choice of the anion. In the case of ipratropium 1h′ the dosages specified above are preferably administered one to four times a day, while administration twice or three times, most preferably three times a day is particularly preferred according to the invention.

Without restricting the scope of the invention thereto, in the case of the cation 1i′ amounts of anticholinergic (1i′) may be administered such that each single dose contains 1000-6500 μg, preferably 2000-6000 μg, particularly preferably 3000-5500 μg, particularly preferably 4000-5000 μg of 1i′. For example and without restricting the present invention thereto, each single dose may contain 3500 μg, 3750 μg, 4000 μg, 4250 μg, 4500 μg, 4750 μg, or 5000 μg of 1i′. The corresponding amount of salt 1i or optionally hydrates or solvates used are easily calculated by the skilled man depending on the choice of the anion. In the case of trospium 1i′ the dosages specified above are preferably administered one to four times a day, while administration twice to three times a day is particularly preferred according to the invention.

Without restricting the scope of the invention thereto, in the case of the compounds 2, quantities of 2′ may be administered such that each single dose contains 1-500 μg, preferably 5-300 μg, particularly preferably 15-200 μg. For example and without restricting the present invention thereto, each single dose may contain 15 μg, 20 μg, 25 μg, ³⁰ μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2′. The corresponding amount of salt 2 or optionally hydrates or solvates used are easily calculated by the skilled man depending on the choice of the anion. In particular in the case of the compound 2a the dosages specified above are preferably administered once to three times a day, while administration once or twice a day is particularly preferred according to the invention.

The medicament combinations of 1 and 2 according to the invention are preferably administered by inhalation. For this reason the ingredients 1 and 2 must be provided in inhalable preparations.

Inhalable preparations include, in particular, inhalable powders. Inhalable powders according to the invention containing the combination of active substances 1 and 2 may consist of the active substances on their own or of a mixture of the active substances with physiologically acceptable excipients. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. Lactose is particularly preferably used as the excipient according to the invention, while lactose monohydrate is most preferably used.

Within the scope of the inhalable powders according to the invention the excipients have a maximum average particle size of up to 250 μm, preferably between 10 and 150 am, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. In particularly preferred inhalable powders the excipient is characterised by a mean particle size of 12 to 35 μm, particularly preferably 13 to 30 μm. Also particularly preferred are those inhalable powders in which the 10% fine fraction is about 1 to 4 μm, preferably about 1.5 to 3 μm in size.

By average particle size is meant here the 50% value of the volume distribution measured with a laser diffractometer using the dry dispersion method. Analogously, the 10% fine content in this instance refers to the 10% value of the volume distribution measured using a laser diffractometer.

Preferably, excipients of high crystallinity are used for the powder formulations according to the invention. This crystallinity can be assessed by means of the enthalpy released as the excipient is dissolved (solution enthalpy). In the case of the excipient lactose monohydrate, which is most preferably used according to the invention, it is preferable to use lactose which is characterised by a solution enthalpy of >45 J/g, preferably >50 J/g, particularly preferably >52 J/g.

Finally, to prepare the inhalable powders according to the invention, micronised active substance 1 and 2, preferably with an average particle size of 0.5 to 10 μm, particularly preferably from 1 to 5 μm, is added to the excipient mixture.

After the starting materials have been weighed in the inhalable powders are prepared from the excipient and the active substances 1 and 2 using methods known in the art. Reference may be made to the disclosure of WO 02/30390, for example.

The inhalable powders according to the invention may be prepared and administered either in the form of a single powder mixture which contains both 1 and 2 or in the form of separate inhalable powders which contain only 1 and 2.

The inhalable powders according to the invention may be administered using inhalers known from the prior art.

Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipient in addition to 1 and 2 are packed into capsules (to produce so-called inhalettes) which are used in inhalers as described, for example, in WO 94/28958.

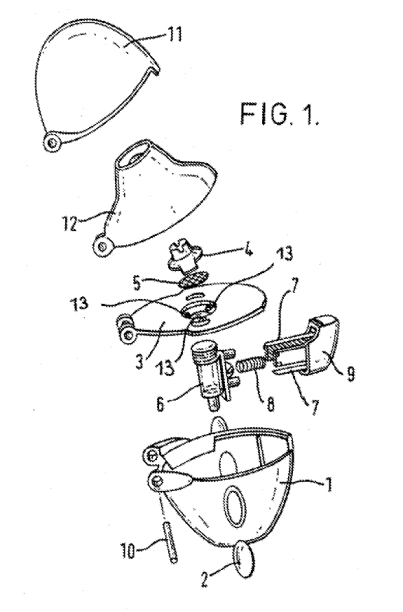

A particularly preferred inhaler for using the pharmaceutical combination according to the invention in inhalettes is shown in FIG. 1.

This inhaler (Handihaler) for inhaling powdered pharmaceutical compositions from capsules is characterised by a housing 1 containing two windows 2, a deck 3 in which there are air inlet openings and which is provided with a screen 5 secured via a screen housing 4, an inhalation chamber 6 connected to the deck 3 on which there is a push button 8 provided with two sharpened pins 7 and movable counter to a spring 8, and a mouthpiece 12 which is connected to the housing 1, the deck 3 and a cover 11 via a spindle 10 to enable it to be flipped open or shut, as well as air holes for adjusting the flow resistance.

For administering the inhalable powders according to the invention containing 1 and 2 using powder-filled capsules it is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 μg/m³, preferably 940-980 μg/m³, more preferably about 960-970 μg/m³ (high density polyethylene).

The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

In another aspect the present invention relates to the abovementioned capsules which contain the abovementioned inhalable powders with 1 and 2 according to the invention. If the inhalable powders according to the invention are to be packed into capsules (inhalettes) as in the preferred use mentioned above, fill levels of 1 to 30 mg, preferably 3 to 20 mg, preferably 5 to 10 mg of inhalable powder per capsule are recommended. These capsules may contain either together or per se the dosages per single dose specified for 1 and 2 above. As already stated, the present invention also relates to a kit consisting of two capsules each of which contains one of the active substances 1 and 2 optionally combined with one of the physiologically acceptable excipients mentioned above.

The present invention also relates to an inhalation kit consisting of one or more of the above capsules characterised by a content of inhalable powder with 1 and 2 according to the invention in conjunction with the inhaler according to FIG. 1.

The present invention also relates to the use of the abovementioned capsules characterised by a content of inhalable powder with 1 and 2 according to the invention, for preparing a pharmaceutical composition for treating respiratory complaints, especially for treating COPD and/or asthma.

Filled capsules which contain the inhalable powders according to the invention are produced by methods known in the art, by filling the empty capsules with the inhalable powders according to the invention.

B) Propellant Gas-Driven Inhalation Aerosols Containing Active Substance Combinations of 1 and 2 According to the Invention

Inhalation aerosols containing propellant gas according to the invention may contain 1 and 2 dissolved in the propellant gas or in dispersed form. 1 and 2 may be present in separate formulations or in a single preparation, in which 1 and 2 are either both dissolved, both dispersed or only one of the components is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a, TG227 and mixtures thereof.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing Active Substance Combinations of 1 and 2 According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the required therapeutic dose within a few seconds to produce an aerosol suitable for therapeutic inhalation. Within the scope of the present invention, preferred nebulisers are those in which a quantity of less than 100 μL, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf in particular FIGS. 6a and 6b). The nebulisers (devices) described therein are known by the name Respimat®.

This nebuliser (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured valve bodies are disclosed for example in WO-94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well, are preferably made of plastics, e.g. by injection moulding. For medicinal purposes, physiologically safe materials are used.

FIGS. 6a/b of WO 97/12687, to which reference is hereby made, show the nebuliser (Respimat®) which can advantageously be used for inhaling the aqueous aerosol preparations according to the invention.

FIG. 6a of WO 97/12687 shows a longitudinal section through the atomiser with the spring biased while FIG. 6b of WO 97/12687 shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

We claim:

1. A pharmaceutical composition comprising an anticholinergic 1 and a pharmacologically acceptable salt of a compound of formula 2′ 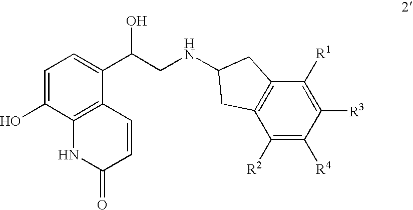
wherein
R¹ and R² which may be identical or different denote hydrogen or C₁-C₄-alkyl;
R³ and R⁴ which may be identical or different denote hydrogen, C₁-C₄-alkyl, —O—C₁-C₄-alkyl, —C₁-C₄-alkylene-O—C₁-C₄-alkyl or
R³ and R⁴ together denote one of the bridging groups —C₁-C₄-alkylene or —O—C₁-C₄-alkylene-O,
wherein the anticholinergic 1 is selected from
a) compounds of formula 1a, 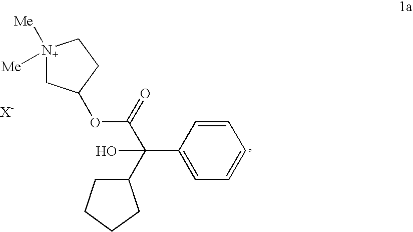
wherein
X⁻ denotes an anion with a single negative charge;
optionally in the form of the diastereomers, mixtures of the diastereomers or racemates thereof, and optionally in the form of the hydrates and/or solvates thereof,
b) compounds of formula 1b 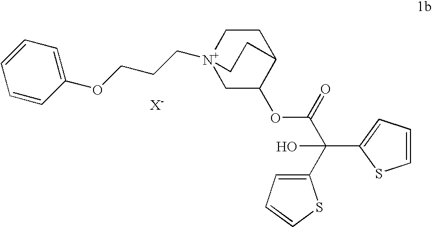
wherein X⁻ is defined above;
optionally in the form of the enantiomers, mixtures of enantiomers or racemates thereof, and optionally in the form of the hydrates and/or solvates thereof,
c) compounds of formula 1c 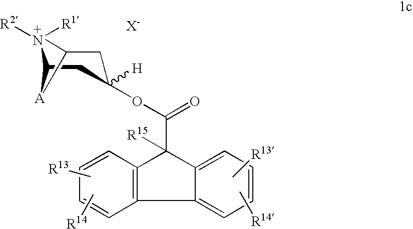
wherein
X⁻ is defined above, and wherein
A denotes a double-bonded group selected from 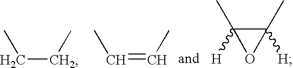
R¹⁵ denotes hydrogen, hydroxy, methyl, ethyl, —CF₃, CHF₂ or fluorine;
R^{1′} and R^{2′} which may be identical or different, denote C₁-C₅-alkyl, optionally substituted by C₃-C₆-cycloalkyl, hydroxy or halogen,
or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen;
d) compounds of formula 1d 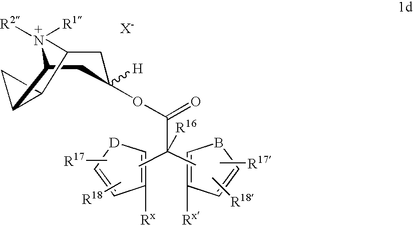
wherein X⁻ is defined above, and wherein
D and B which may be identical or different, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, optionally substituted by —C₃-C₆-cycloalkyl, hydroxy or halogen,
or
R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen,
or
R^{x} and R^{x′} together denote a single bond or a double-bonded group selected from O, S, NH, CH₂, CH₂—CH₂, N(C₁-C₄-alkyl), CH(C₁-C₄-alkyl) and —C(C₁-C₄-alkyl)₂;
e) compounds of formula 1e 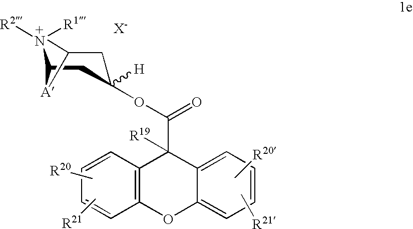
wherein X⁻ is defined above, and wherein
A′ denotes a double-bonded group selected from 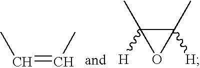
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, optionally substituted by C₃-C₆-cycloalkyl, hydroxy or halogen,
or
R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen; and
f) oxitropium salts (1f), flutropium salts (1g), ipratropium salts (1h) and trospium salts (1i),
optionally in combination with pharmaceutically acceptable excipients.

2. The pharmaceutical composition according to claim 1, wherein X— is selected from fluoride, chloride, bromide, iodide, sulphate, phosphate, methanesulphonate, nitrate, maleate, acetate, citrate, fumarate, tartrate, oxalate, succinate, benzoate and p-toluenesulphonate, optionally in the form of the racemates, enantiomers or hydrates thereof.

3. The pharmaceutical composition according to claim 1, wherein in 2′,
R¹ and R² which may be identical or different denote hydrogen, methyl or ethyl;
R³ and R⁴ which may be identical or different denote hydrogen, methyl, ethyl, propyl, butyl, methoxy, ethoxy, methyoxymethyl, or methoxyethyl, or
R³ and R⁴ together denote one of the bridging groups propylene, butylene, —O-ethylene-O or —O-propylene-O—.

4. The pharmaceutical composition according to claim 1 or 3, wherein 1 and 2′ are either present together in a single preparation or are contained in two separate preparations.

5. The pharmaceutical composition according to claim 1 or 3, wherein 1 and 2′ are in a weight ratio of from about 1:30 to about 400:1.

6. The pharmaceutical composition according to claim 1 or 3, wherein 1 and 2′ are in a weight ratio of from about 1:25 to about 200:1.

7. The pharmaceutical composition according to claim 1, wherein the pharmaceutical composition is administered to a patient as a single dose of 0.01 μg to 10000 μg.

8. The pharmaceutical composition according to claim 7, wherein the pharmaceutical composition is administered to a patient as a single dose of 0.1 μg to 5000 μg.

9. The pharmaceutical composition according to claim 1, wherein the pharmaceutical composition is administered by inhalation.

10. The pharmaceutical composition according to claim 9, wherein the pharmaceutical composition is an inhalable powder, a propellant-containing metered dose aerosol, or a propellant-free inhalable solution or suspension.

11. The pharmaceutical composition according to claim 10, wherein the pharmaceutical composition is an inhalable powder comprising 1 and 2′ in admixture with a suitable physiologically acceptable excipient selected from monosaccharides, disaccharides, oligo- and polysaccharides, polyalcohols, salts, and mixtures thereof.

12. The pharmaceutical composition according to claim 11, wherein the excipient has a maximum mean particle size of 250 μm or less.

13. The pharmaceutical composition according to claim 11, wherein the excipient has a maximum mean particle size of between 10 μm and 150 μm.

14. The pharmaceutical composition according to claim 10, wherein the inhalable powder contains active substances 1 and 2′.

15. The pharmaceutical composition according to claim 10, wherein the propellant-containing inhalable aerosol which comprise 1 and 2′ in dissolved or dispersed form.

16. The pharmaceutical composition according to claim 10, wherein the propellant-free inhalable solution or suspension comprises water, ethanol or mixtures thereof as solvent.

17. A method of treating inflammatory or obstructive respiratory complaints comprising administering to a patient in need thereof the composition according to claim 1.
